(19) United States
(12) Patent Application Publication
(10) Pub. No.: US 2007/0197909 A1
(43) Pub. Date: Aug. 23, 2007

(54) System and method for displaying image studies using hanging protocols with perspectives/views

(75) Inventors: Murali Kumaran Kariathungal, Hoffman Estates, IL (US); Mark Ricard, Naperville, IL (US); James Jay, Palatine, IL (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/347,917

(22) Filed: Feb. 6, 2006

(51) Int. Cl. A61B 8/00

(52) U.S. Cl. 600/437

(57) ABSTRACT

Certain embodiments of the present invention provide methods and systems for displaying images using a plurality of perspectives within a hanging protocol. Certain embodiments include loading a hanging protocol for display of an image study, selecting a perspective within the hanging protocol for display of the image study, and displaying the image study based on a display configuration within the selected perspective. In certain embodiments, the perspective is selected from a plurality of perspectives, including a default perspective, within the hanging protocol. In certain embodiments, selection of the perspective comprises selection of a perspective based on modality, anatomy, procedure, and/or default view, for example. In certain embodiments, a perspective may be automatically within the hanging protocol for display of the image study. In certain embodiments, a user may be allowed to switch perspectives during display of the image study.

### BACKGROUND OF THE INVENTION

The present invention generally relates to dynamic display configuration in a picture archiving and communication system. In particular, certain embodiments of the present invention relate to dynamic configuration and management of applications across a plurality of displays in a picture archiving and communication system.

A clinical or healthcare environment is a crowded, demanding environment that would benefit from organization and improved ease of use of imaging systems, data storage systems, and other equipment used in the healthcare environment. A healthcare environment, such as a hospital or clinic, encompasses a large array of professionals, patients, and equipment. Personnel in a healthcare facility must manage a plurality of patients, systems, and tasks to provide quality service to patients. Healthcare personnel may encounter many difficulties or obstacles in their workflow.

A variety of distractions in a clinical environment may frequently interrupt medical personnel or interfere with their job performance. Furthermore, workspaces, such as a radiology workspace, may become cluttered with a variety of monitors, data input devices, data storage devices, and communication device, for example. Cluttered workspaces may result in efficient workflow and service to clients, which may impact a patient's health and safety or result in liability for a healthcare facility. Data entry and access is also complicated in a typical healthcare facility.

Healthcare environments, such as hospitals or clinics, include clinical information systems, such as hospital information systems (HIS) and radiology information systems (RIS), and storage systems, such as picture archiving and communication systems (PACS). Information stored may include patient medical histories, imaging data, test results, diagnosis information, management information, and/or scheduling information, for example. The information may be centrally stored or divided at a plurality of locations. Healthcare practitioners may desire to access patient information or other information at various points in a healthcare workflow. For example, during surgery, medical personnel may access patient information, such as images of a patient's anatomy, that are stored in a medical information system. Alternatively, medical personnel may enter new information, such as history, diagnostic, or treatment information, into a medical information system during an ongoing medical procedure.

A reading, such as a radiology or cardiology procedure reading, is a process of a healthcare practitioner, such as a radiologist or a cardiologist, viewing digital images of a patient. The practitioner performs a diagnosis based on a content of the diagnostic images and reports on results electronically (e.g., using dictation or otherwise) or on paper. The practitioner, such as a radiologist or cardiologist, typically uses other tools to perform diagnosis. Some examples of other tools are prior and related prior (historical) exams and their results, laboratory exams (such as blood work), allergies, pathology results, medication, alerts, document images, and other tools.

Picture archiving and communication systems (“PACS”) connect to medical diagnostic imaging devices and employ an acquisition gateway (between the acquisition device and the PACS), storage and archiving units, display workstations, databases, and sophisticated data processors. These components are integrated together by a communication network and data management system. A PACS has, in general, the overall goals of streamlining health-care operations, facilitating distributed remote examination and diagnosis, and improving patient care.

A typical application of a PACS system is to provide one or more medical images for examination by a medical professional. For example, a PACS system can provide a series of x-ray images to a display workstation where the images are displayed for a radiologist to perform a diagnostic examination. Based on the presentation of these images, the radiologist can provide a diagnosis. For example, the radiologist can diagnose a tumor or lesion in x-ray images of a patient's lungs.

Current PACS systems use general techniques known as “hanging protocols” to format display or layout of images. Hanging protocols allow a user to display images based on modality, anatomy, and procedure. Hanging protocols present a perspective or view to a user, such as a radiologist. Images may be grouped according to characteristics such as DICOM series or series number.

Additionally, PACS systems attempt to prepare images for viewing by users by applying a series of processing steps or functions included in a Default Display Protocol (“DDP”). A DDP is a default workflow that applies a series of image processing functions to image data to prepare the image data for presentation to a user on a particular monitor configuration. DDPs typically include processing steps or functions that are applied before any diagnostic examination of the images. A DDP may be based on a type of imaging modality used to obtain the image data, for example. In general, a DDP attempts to present image data in a manner most useful to many users.

With increasing volumes of examinations and images, a reduction of radiologists and mounting pressures on improved productivity, radiologists and other healthcare personnel are in need of image processing or display workflow enhancements that alleviate rote, repetitive tasks. Such enhancements may include the dynamic modification of DDPs so as to incorporate processing functions routinely selected by a radiologist, for example. In other words, improved DDPs that may be modified to incorporate a processing function frequently selected by a given radiologist for a certain type of image, for example, would be highly desirable. The modified DDP may then automatically process subsequent image data according to the processing functions routinely selected by the radiologist. The radiologist would not have to manually select the addition of processing steps as they have been incorporated into the DDP. However, current PACS systems do not provide for such enhancements.

Currently, when a user works on a multiple monitor system, the user may not work on multiple applications if a PACS workflow application, such as Centricity PACS, is running because the PACS workflow application occupies all the monitors and occludes other applications currently displayed on different monitors connected to the system. Such a behavior may be a limiting factor for a user who is working with multiple applications along with a PACS workstation. Additionally, it is highly desirable that none of the PACS features are affected. For example, a user wishes to use a PACS workflow engine, such as Centricity™ PACS, with ancillary programs, such as surgical planning tools and/or image processing tools, open. Alternatively, a user wishes to work on Centricity™ PACS while referring to a related e-mail in his/her Microsoft Outlook® or other e-mail viewing program, for example. Additionally, a user may want to work on a PACS application while referring to an article on the Internet or other network, for example.

In order to display a study with different views (such as with historicals, without historicals, etc.), a user must create different protocols under PACS workstations. Additionally, a user may use only one protocol at any time when opening a study. In order to use other protocols, the user must apply or select a different protocol.

Thus, there is a need for a system and method for dynamic display configuration in a PACS environment. There is a need for a system and method allowing a user to switch views for easy reading of an image study.

### BRIEF SUMMARY OF THE INVENTION

Certain embodiments of the present invention provide methods and systems for displaying images using a plurality of perspectives within a hanging protocol. Certain embodiments provide a method for displaying an image studying using perspectives. The method includes loading a hanging protocol for display of an image study, selecting a perspective within the hanging protocol for display of the image study, and displaying the image study based on a display configuration within the selected perspective.

In certain embodiments, the perspective is selected from a plurality of perspectives, including a default perspective, within the hanging protocol. In certain embodiments, selection of the perspective comprises selection of a perspective based on modality, anatomy, procedure, and/or default view, for example. In certain embodiments, the step of selecting further includes automatically selecting a perspective within the hanging protocol for display of the image study. In certain embodiments, a user may be allowed to switch perspectives during display of the image study. In certain embodiments, the method further includes dynamically adjusting a perspective based on the display configuration.

Certain embodiments provide a display system configured to display images on a display. The system includes a hanging protocol for display of images according to a plurality of display configurations, and a plurality of perspectives associated with the hanging protocol. Each of the perspectives is associated with at least one display configuration for display of images on a display, and one of the plurality of perspectives is selected from within the hanging protocol for display of the images.

In certain embodiments, one of the plurality of perspectives is automatically selected from within the hanging protocol for display of the images. In certain embodiments, the plurality of perspectives includes a default perspective. In certain embodiments, selection of the perspective comprises selection of a perspective based on modality, anatomy, procedure, and/or default view, for example. In certain embodiments, the selected perspective is capable of being switched during image display.

Certain embodiments provide a method for flexible display configuration. The method includes defining a hanging protocol capable of displaying image data on a display under a plurality of display configurations, and determining a plurality of perspectives for the plurality of display configurations. The perspectives facilitate display of image data on the display based on the display configurations.

In certain embodiments, the method also includes designating a default perspective for default display of image data. In certain embodiments, the method further includes configuring automatic selection of a perspective for display of image data based on at least one of modality, anatomy, and procedure, for example. In certain embodiments, the method includes saving the hanging protocol with the plurality of perspectives for selection by a user to display image data.

Certain embodiments provide a computer-readable storage medium including a set of instructions for execution on a processor. The set of instructions includes a selection routine for selecting one of a plurality of perspectives within a hanging protocol for display of image data on a display, and a display routine for configuring the display for display of the image data according to the selected perspective within the hanging protocol.

In certain embodiments, the perspective is selected from a plurality of perspectives, including a default perspective, within the hanging protocol. In certain embodiments, selection of the perspective includes selection of a perspective based on at least one of modality, anatomy, procedure, and default view, for example. In certain embodiments, the selection routine automatically selects a perspective within the hanging protocol for display of the image data. In certain embodiments, the selection routine and the display routine allow a perspective to be switched during display of the image data.

### BRIEF DESCRIPTION OF SEVERAL VIEWS OF THE DRAWINGS

FIG. 1 illustrates an exemplary picture archiving and communication system used in accordance with an embodiment of the present invention.

FIG. 2 shows examples of perspectives.

FIG. 3 illustrates a flow diagram for a method for configuration of hanging protocols and related perspectives in accordance with an embodiment of the present invention.

FIG. 4 illustrates a flow diagram for a method for displaying an image study using a display protocol having multiple perspectives used in accordance with an embodiment of the present invention.

FIG. 5 shows an example of display of an image series from a current study using a single hanging protocol in a two-monitor configuration with a perspective including no historical images in a default view in accordance with an embodiment of the present invention.

FIG. 6 depicts an exemplary view of a perspective showing a comparison between a current image series and a historical image series in accordance with an embodiment of the present invention.

FIG. 7 shows an example of a perspective with image stacks for two series in a current image study without historical image comparison in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

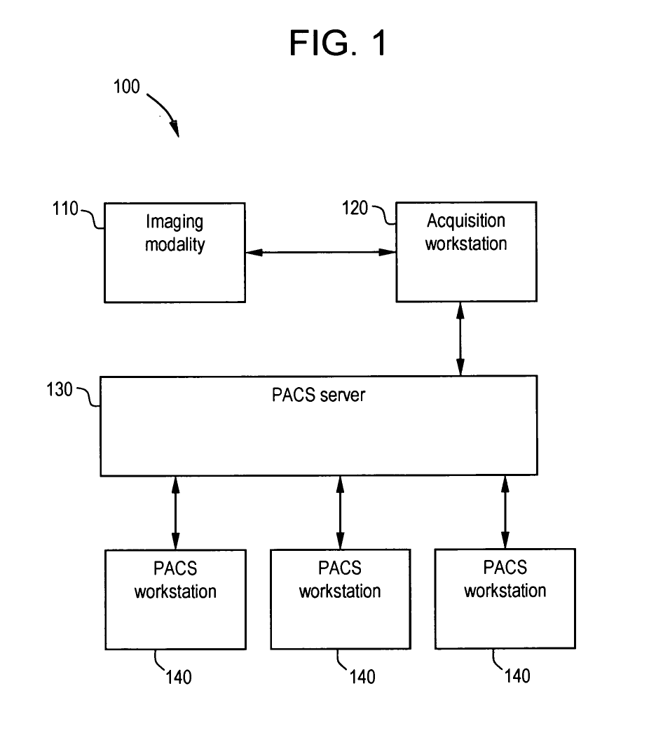

FIG. 1 illustrates an exemplary Picture Archiving and Communication System (PACS) 100 used in accordance with an embodiment of the present invention. The PACS system 100 includes an imaging modality 110, an acquisition workstation 120, a PACS server 130, and one or more PACS workstations 140. The system 100 may include any number of imaging modalities 110, acquisition workstations 120, PACS server 130 and PACS workstations 140 and is not in any way limited to the embodiment of system 100 illustrated in FIG. 1. The components of the system 100 may communicate via wired and/or wireless communication, for example, and may be separate systems and/or integrated to varying degrees, for example.

In operation, the imaging modality 110 obtains one or more images of a patient anatomy. The imaging modality 110 may include any device capable of capturing an image of a patient anatomy such as a medical diagnostic imaging device. For example, the imaging modality 110 may include an X-ray imager, ultrasound scanner, magnetic resonance imager, or the like. Image data representative of the image(s) is communicated between the imaging modality 110 and the acquisition workstation 120. The image data may be communicated electronically over a wired or wireless connection, for example.

In an embodiment, the acquisition workstation 120 may apply one or more preprocessing functions, for example, to the image data in order to prepare the image for viewing on a PACS workstation 140. For example, the acquisition workstation 120 may convert raw image data into a DICOM standard format or attach a DICOM header. Preprocessing functions may be characterized as modality-specific enhancements, for example (e.g., contrast or frequency compensation functions specific to a particular X-ray imaging device), applied at the beginning of an imaging and display workflow. The preprocessing functions differ from processing functions applied to image data in that the processing functions are not modality specific and are instead applied at the end of the imaging and display workflow (for example, at a display workstation 140).

The image data may then be communicated between the acquisition workstation 120 and the PACS server 130. The image data may be communicated electronically over a wired or wireless connection, for example.

The PACS server 130 may include computer-readable storage media suitable for storing the image data for later retrieval and viewing at a PACS workstation 140. The PACS server 130 may also include one or more software applications for additional processing and/or preprocessing of the image data by one or more PACS workstations 140.

One or more PACS workstations 140 are capable of or configured to communicate with the server 130. The PACS workstations 140 may include a general purpose processing circuit, a PACS server 130 interface, a software memory, and/or an image display monitor, for example. The PACS server 130 interface may be implemented as a network card connecting to a TCP/IP based network, but may also be implemented as a parallel port interface, for example.

The PACS workstations 140 may retrieve or receive image data from the server 130 for display to one or more users. For example, a PACS workstation 140 may retrieve or receive image data representative of a computed radiography (CR) image of a patient's chest. A radiologist or user may then examine the image for any objects of interest, such as tumors, lesions, etc., for example.

The PACS workstations 140 may also be capable of or configured to apply processing functions to image data. For example, a user may desire to apply processing functions to enhance features within an image representative of the image data. Processing functions may therefore adjust an image of a patient anatomy in order to ease a user's diagnosis of the image. Such processing functions may include any software-based application that may alter a visual appearance or representation of image data. For example, a processing function can include any one or more of flipping an image, zooming in an image, panning across an image, altering a window and/or level in a grayscale representation of the image data, and altering a contrast and/or brightness an image.

In an embodiment, the PACS system 100 may provide one or more perspectives for viewing images and/or accessing applications at a PACS workstation 140. Perspectives may be provided locally at the PACS workstation 140 and/or remotely from the PACS server 130. In an embodiment, the PACS system 100 includes a perspectives manager capable of being used for reviewing images via a plurality of perspectives. The PACS server 130 and/or a PACS workstation 140 may include the perspectives manager, or the perspectives manager may be implemented in a separate system. In an embodiment, each PACS workstation 140 may include a perspectives manager.

In operation, for example, a user, such as a radiologist, selects a set of images, such as screening mammogram images, chest screening images and/or other computed radiography (CR), digital radiography (DR), and/or digital x-ray (DX) screening images, to review at a PACS workstation 140. The images may be displayed in a default perspective and/or a customized perspective, for example.

As described above, a user may wish to apply additional processing steps to one or more images to further enhance features in the image. For example, a user may desire to apply additional processing functions or steps to an image in order to alter the presentation of an image in conformance with the user's confidence level for making an accurate diagnosis. In other words, different users may desire to apply different or additional processing steps than are included in a default image processing workflow.

The additional image processing step(s) may include any image processing step useful to prepare an image for a diagnostic examination. For example, as described above, an image processing step (as a default image processing step or an additional image processing step) can include flipping an image, zooming in an image, panning across an image, and altering one or more of a window, a level, a brightness and a contrast setting of an image. Image data may be displayed on a PACS workstation 140 using the same and/or different processing, display protocol, and/or perspective as other image(s), for example.

PACS workstations 140 may retrieve or receive image data from server 130 for display to one or more users. For example, a PACS workstation 140 may retrieve or receive image data representative of a computed radiography image of a patient's chest. A radiologist may then examine the image as displayed on a display device for any objects of interest such as, for example, tumors, lesions, etc.

PACS workstations 140 are also capable of or configured to retrieve and/or receive one or more hanging protocols from server 130. For example, a default hanging protocol may be communicated to PACS workstation 140 from server 130. A hanging protocol may be communicated between server 130 and a PACS workstation 140 over a wired or wireless connection, for example.

In general, PACS workstations 140 may present images representative of image data retrieved and/or received from server 130. PACS workstations 140 may present the images according to a hanging protocol. As described above, a hanging protocol is a set of display rules for presenting, formatting and otherwise organizing images on a display device of a PACS workstation 140. A display rule is a convention for presenting one or more images in a particular temporal and/or spatial layout or sequence. For example, a hanging protocol may include a set of computer-readable instructions (or display rules, for example) that direct a computer to display a plurality of images in certain locations on a display device and/or display the plurality of images in a certain sequence or order. In another example, a hanging protocol may include a set of computer-readable instructions that direct a computer to place a plurality of images in multiple screens and/or viewports on a display device. In general, a hanging protocol may be employed to present a plurality of images for a diagnostic examination of a patient anatomy featured in the images.

A hanging protocol may direct, for example, a PACS workstation 140 to display an anterior-posterior (“AP”) image adjacent to a lateral image of the same anatomy. In another example, a hanging protocol may direct PACS workstation 140 to display the AP image before displaying the lateral image. In general, a hanging protocol dictates the spatial and/or temporal presentation of a plurality of images at PACS workstation 140.

A hanging protocol differs from a default display protocol (“DDP”). In general, a DDP is a default workflow that applies a series of image processing functions to image data. The image processing functions are applied to the image data in order to present an image (based on the image data) to a user. The image processing functions alter the appearance of image data. For example, an image processing function may alter the contrast level of an image.

DDPs typically include processing steps or functions that are applied before any diagnostic examination of the images. For example, processing functions may be applied to image data in order to enhance features within an image (based on the image data). Such processing functions can include any software-based application that may alter a visual appearance or representation of image data. For example, a processing function can include any one or more of flipping an image, zooming in an image, panning across an image, altering a window and/or level setting in a representation of the image data, and altering a contrast and/or brightness setting in a representation of the image data.

DDPs are usually based on a type of imaging modality used to obtain the image data. For example, image data obtained with a C-arm imaging device in general or a particular C-arm imaging device may have a same or similar DDP applied to the image data. In general, a DDP attempts to present image data in a manner most useful to many users.

Conversely, applying a hanging protocol to image data does not alter the appearance of an image (based on the image data), but instead dictates how the image(s) is (are) presented, as described above.

Server 130 may store a plurality of hanging protocols and/or DDPs. The hanging protocols and/or DDPs that are stored at server 130 and have not yet been modified or customized are default hanging protocols/DDPs. A default hanging protocol and/or DDP may be selected from a plurality of default hanging protocols and/or DDPs based on any number of relevant factors such as, for example, a manual selection, a user identity, and/or pre-processing of the image data.

Specifically, a default hanging protocol and/or DDP may be selected based on a manual selection simply by communicating the default protocol once a user has selected that particular protocol. The user may make the selection, for example, at a PACS workstation 140.

In another example, a default protocol may be selected based on a user identity. For example, a user may have a preferred DDP. The DDP may have been customized to meet the user's preferences for a particular temporal and/or spatial layout of images. Once a user gains access to a PACS workstation 140 (for example, by entering a correct login and password combination or some other type of user identification procedure), the preferred DDP may be communicated to the PACS workstation 140, for example.

In another example, a default protocol may be selected based on pre-processing of image data. Pre-processing of image data may include any image processing known to those of ordinary skill in the art that prepares an image for review by a user. Pre-processing may also include, for example, a computer-aided diagnosis (“CAD”) of image data. CAD of image data may include a computer (or similar operating unit) automatically analyzing image data for objects of interest. For example, a CAD may include a software application that analyzes image data for nodules in images of lungs, lesions, tumors, etc. However, a CAD application can include any automatic analysis of image data known to those of ordinary skill in the art.

For example, a default hanging protocol that corresponds to CAD findings of lung tumors may provide for the presentation of the posterior-anterior (PA) and lateral lung images adjacent to each other followed by the presentation of the computer tomography (CT) lung images, followed by the magnetic resonance (MR) lung images, for example. In general, a default hanging protocol that corresponds to CAD findings is designed to present images in a spatial and/or temporal layout that is useful to a radiologist. For example, a radiologist may be greatly assisted in his or her review of the CAD findings by viewing the PA and lateral lung images adjacent to each other, followed by previously acquired multi-slice CT and MR images of the lungs.

Therefore, based on CAD findings, a default protocol may be selected from a plurality of default protocols and applied at a workstation 140 in order to present images to a user.

PACS users often wish to run multiple applications on a PACS workstation 140. In addition to a primary PACS workflow or interface application, a user may wish to access other applications such as surgical planning tools, scheduling tools, electronic mail viewers, image processing tools, and/or other tools. For example, PACS users often like to use a PACS workflow engine while viewing electronic mail and accessing information on the Internet. Users of an integrated RIS/PACS system may wish to access both RIS and PACS applications simultaneously. Typically, however, the PACS application occupies all active display area and hides other applications running on the workstation 140. For example, in a PACS workstation 140 having three monitors, the PACS workflow application occupies all three monitors. When an application is initiated, another application may be displaced, or the application may be launched in a sub-optimal display area. For example, a user may launch a data management or diagnostic processing software at a three-monitor PACS workstation 140, and the application may launch on a color monitor, displacing images displayed on the color monitor. Typically, a user would have to manually reorganize applications to display the management application on a grayscale monitor and the images on the higher resolution color monitor.

Certain embodiments provide an adaptable PACS system 100 accommodating a plurality of displays such that each display operates with a separate display window. All display windows are controlled internally by a primary window that is transparent to users. The primary, transparent window tracks which window or windows have the PACS application and which window(s) have other applications and/or data. Thus, the PACS application and other applications may be simultaneously displayed on a plurality of displays.

Certain embodiments provide dynamic configuration of displays associated with PACS workstation 140. The primary window allows interaction or application(s) and data across multiple windows. The PACS workstation 140 operates a transparent, primary window including a plurality of windows across a plurality of displays.

Selection of a hanging protocol on a PACS workstation may be based on a plurality of criteria, such as a number of connected displays, a modality, an anatomy, and a procedure, for example. Based on these criteria, a user may create multiple protocols with one default protocol used to display an image study. For example, a hanging protocol may be created for a particular display configuration. A user creates different hanging protocols to properly display a study on different display configurations.

However, certain embodiments allow creation of a protocol including a plurality of perspectives or views, for example. Using one protocol with multiple perspectives/views, a user may associate different perspectives/views for different display configurations with the protocol. For example, a hanging protocol may include multiple perspectives with one default perspective. The default perspective may be used to display a study unless otherwise specified and/or determined manually or automatically, for example.

In certain embodiments, hanging protocols with perspectives/views may use one or more criteria to select a protocol for display. For example, a modality, an anatomy or body part, a procedure, and/or a default view for a display configuration, may be used to select an appropriate display protocol. For example, a display protocol includes a perspective/view with multiple options depending upon monitor configuration. A user may create a hanging protocol with different view for different display configurations, for example. A user does not have to create different hanging protocols for different monitor configurations but may instead create additional views with the existing hanging protocol. In certain embodiments, a user may switch between different perspectives/views after opening a study.

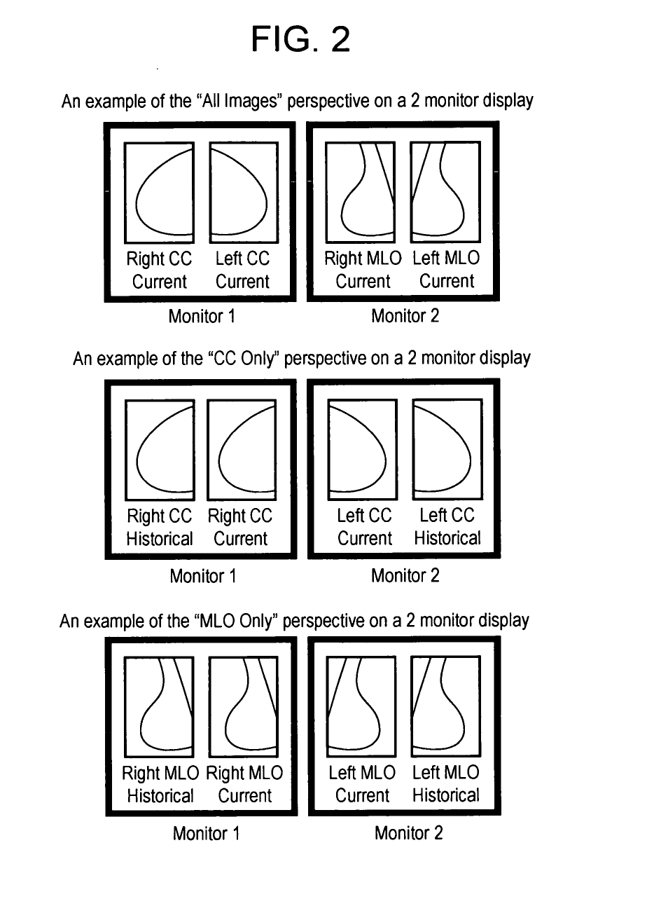

In certain embodiments, perspectives are views or layouts indicating visual component positioning and interactions between images and/or applications based on workflow, for example. Medical perspectives may be used to create a plurality of benefits for. For example, perspectives may provide patient context sharing between different image(s) and/or application(s) that a user views. Additionally, for example, perspectives provide an ability to easily switch between different configurations or perspectives based on which images and/or applications a user wishes to view at any given point. Furthermore, for example, perspectives provide an ability to store or “remember” specific workflow steps. Perspectives provide a mechanism to save and display information relevant to a particular user, group, and/or function, for example. Perspectives may be used to display images and other data for a particular resolution, display type, and/or other configuration, for example. Some examples of perspectives are depicted in FIG. 2.

Perspectives may be used to logically group different images and/or other data or applications. For example, perspectives may be defined for images, examination results, laboratory data, patient history data, structured report data, DICOM data, and/or other data or applications, for example. Rules, configuration options, and/or other criteria may be defined in order to define perspectives. In certain embodiments, perspectives do not eliminate or change information but rather order information in a certain way. For example, information important to a user may be displayed first, with additional information available via different perspectives. In certain embodiments, perspectives may be created automatically based on user selection or other configuration information, for example. In certain embodiments, a perspective may work together with a rules-based context manager to filter and display information.

In certain embodiments, medical are software components that save visual component positioning and interactions between medical applications and data based on workflow. Medical application perspectives are a mechanism used to create a plurality of benefits for users. For example, perspectives may provide patient context sharing between different applications, data and/or other components that a user views. Additionally, for example, perspectives provide an ability to switch between different configurations or perspectives based on which applications, data and/or other components a user wishes to view at any given point. Furthermore, for example, perspectives provide an ability to store or “remember” specific workflow steps. Perspectives provide a mechanism to save and display information relevant to a particular user, group, and/or function, for example.

Perspectives that may be saved by and/or for one or more users. For example, a perspective may include viewing an exam worklist on a color monitor, one or more images displayed on one or more diagnostic monitors, and a report editor on the bottom of the color monitor. For example, another perspective may include viewing related prior report(s) on the color monitor, related prior image(s) on one diagnostic monitor, and current image(s) on another diagnostic monitor. For example, a perspective may show viewing all labs and allergies for a period of time (e.g., two months) for a patient on the color monitor and viewing current image(s) on the diagnostic monitor(s). As another example, a perspective may include viewing any maximum intensity projection/multiplanar reconstruction (MIP/MPR) image set for a current exam on a diagnostic monitor.

In certain embodiments, users may “switch to” or “be assigned” a medical perspective on the fly. Based on available perspectives, a user may toggle between perspectives to read an exam or other data. A user may toggle between available perspectives using a mousing device, keyboard shortcuts, gaze tracking, and/or voice command, for example. In certain embodiments, specific workflows of individual radiologists and/or cardiologists may be stored so that each radiologists/cardiologists uses the same workflow through the same sequence of perspectives wherever the user logs in. Thus, a user has the advantage of reading exams and other data quickly and efficiently on any diagnostic workstation, for example.

In certain embodiments, software, firmware and/or hardware may verify a user's right to access one or more of the applications and/or perspectives. For example, if a user logs on to a system with perspectives, based on previous saving of a default perspective, the user is logged on automatically into RIS, PACS, and EMR systems.

Thus, a user may access relevant prior history for a patient (e.g., images and reports). Using different perspectives the user has already created, the user may switch between perspectives to view desired information. The medical application perspectives may be delivered to the user in a variety of ways. For example, perspectives may be delivered via a preselected set of components and/or workflows from a medical software and/or hardware provider. Perspectives may also be delivered via perspectives created by a system administrator. Additionally, a user may dynamically create perspectives during operation of the system 100 (i.e., “on the fly”). Thus, the user may select components and/or applications for display in viewable areas of one or more monitors based on workflow. The information/configuration may then be saved in one or more perspectives. The user may toggle between perspectives to read an exam or other data on a variety of devices such as displays and/or printers. The user may save perspectives, exams, reports, and/or other data, for example.

In an embodiment, a plurality of applications may be providing information to a radiologist, cardiologist and/or other user for diagnosis of a patient. One or more displays available to the user may not have enough screen space to display all of the information. Additionally, displaying all of the available information would be too crowded to be useful. Even if information is filtered with rules, too much information may still remain. Thus, a user may apply medical perspectives on a workstation to view information from a plurality of applications and systems. One perspective may be set up to show images and/or examination results from radiology, for example. Another perspective may be set up to show images and/or examination results from cardiology, for example. Another perspective may be set up to show images and/or examination results from imaging, for example.

Perspectives may be used to logically group different applications, for example. Rules, configuration options, and/or other criteria may be defined in order to define perspectives. Perspectives may be defined for images, examination results, laboratory data, patient history data, structured report data, DICOM data, and/or other data, for example. In certain embodiments, perspectives do not eliminate or change information but rather order information in a certain way. For example, information important to a user may be displayed first, with additional information available via different perspectives. In certain embodiments, a system may “learn” through user selection or other configuration information, for example, to create perspectives automatically without manual intervention by the user.

In certain embodiments, images in perspectives may be organized according to one or more criterion. For example, the default perspective includes a first set of images organized according to a default criterion, while the second perspective includes a second set of images organized according to a second criterion. The second criterion may be different from the default criterion, for example. Additionally, the second set of images may be a subset of the first or default set of images, which may include all available images for a subject, procedure, modality, and/or user, for example.

In certain embodiments, the criterion includes image attributes, such as procedure-specific image attributes. For example, the second perspective may be organized or laid out based on mammogram-specific image attributes found in image DICOM headers. Use of image attributes in determining a perspective layout allows precision in reproducing a perspective for each instance of a procedure, for example.

Additionally, a display protocol, such as a Default Display Protocol (DDP) may be adjusted for one or more displays based on content and/or a number of connected display(s). For example, if the PACS workstation 140 is reconfigured from a three monitor configuration to a one monitor configuration, the DDP may be modified accordingly. Certain embodiments adapt a DDP based on application(s) closed and/or opened as well as window(s) activated and/or deactivated. For example, a DDP may determine what information is displayed to a user. A DDP may adapt based on a number of available monitors and a number of images to be displayed, for example (e.g., four images are shown on one available display; eight images are shown on two available displays, etc). PACS workstation 140 may configure a DDP for any multi-monitor full screen and/or partial screen applications. Additionally, one or more applications may be resized on a single screen (e.g., minimize, maximize, and/or resize).

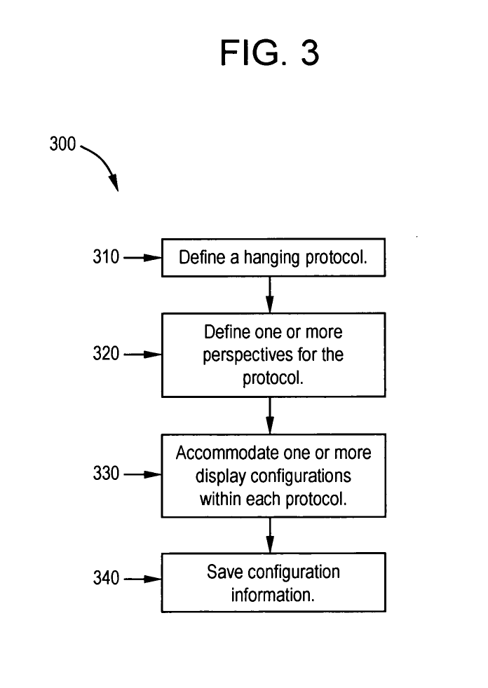

FIG. 3 illustrates a flow diagram for a method 300 for configuration of hanging protocols and related perspectives in accordance with an embodiment of the present invention. First, at step 310, a hanging or display protocol is defined. Then, at step 320, one or more perspectives/views are defined for that protocol. Next, at step 330, one or more display configurations may be accommodated within each perspective/view. At step 340, configuration information regarding a hanging protocol and its associated perspective(s) and display configuration(s) may be saved for later use. Alternatively or in addition, the configuration information may be used for display of content without saving the configuration information. In certain embodiments, a perspective may be defined as a default perspective for one or more display configurations.

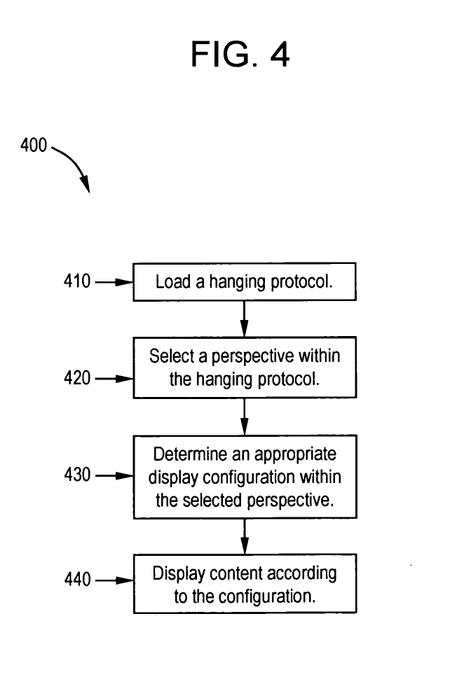

FIG. 4 illustrates a flow diagram for a method 400 for displaying an image study using a display protocol having multiple perspectives used in accordance with an embodiment of the present invention. How configure, then how use. First, at step 410, a hanging or display protocol is loaded. Then, at step 420, a perspective or view is selected. Next, at step 430, an appropriate display configuration is determined within the selected perspective. The display configuration may be determined manually by a user and/or automatically by a software, hardware and/or firmware system, for example. At step 440, content is displayed according to the configuration.

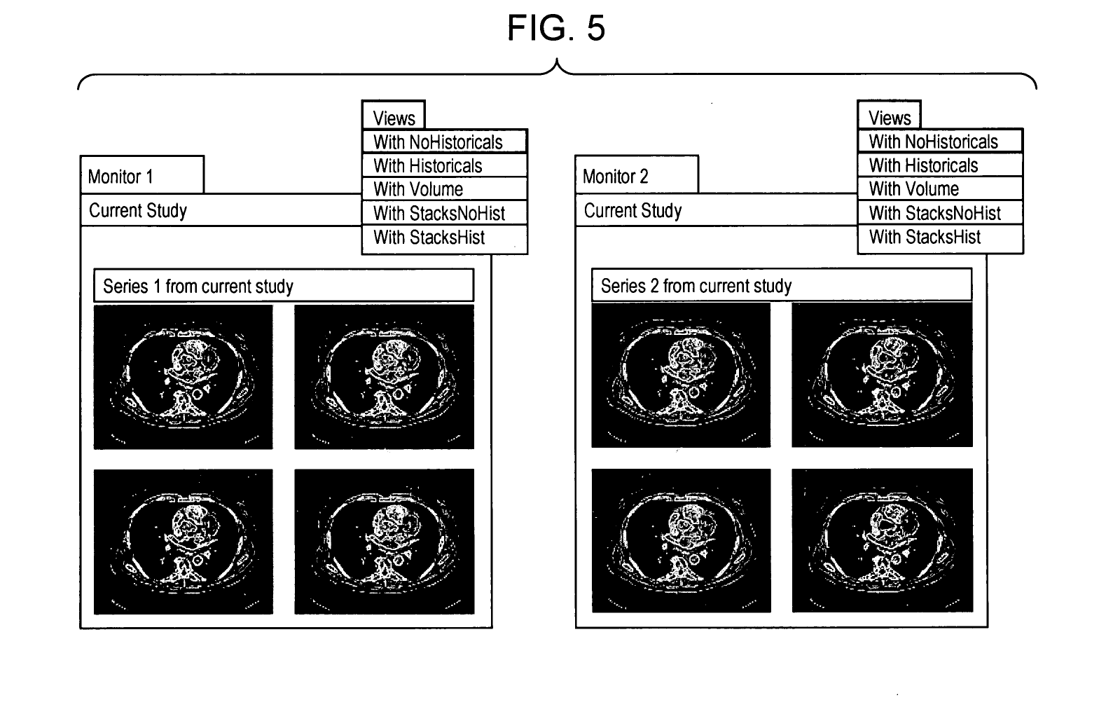

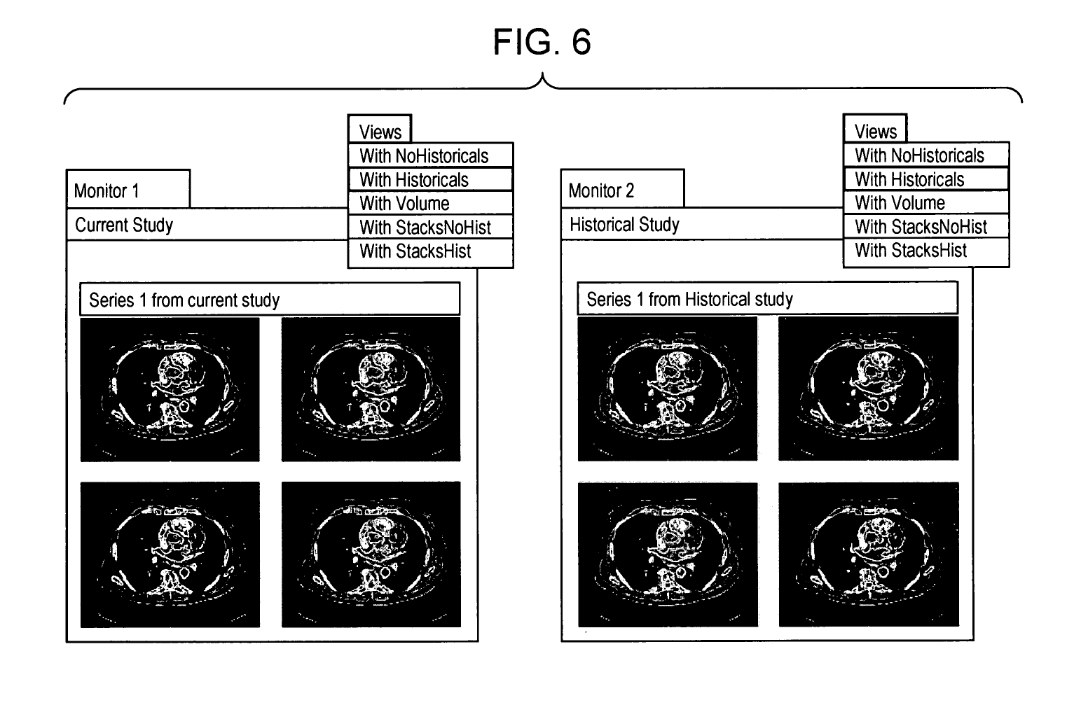

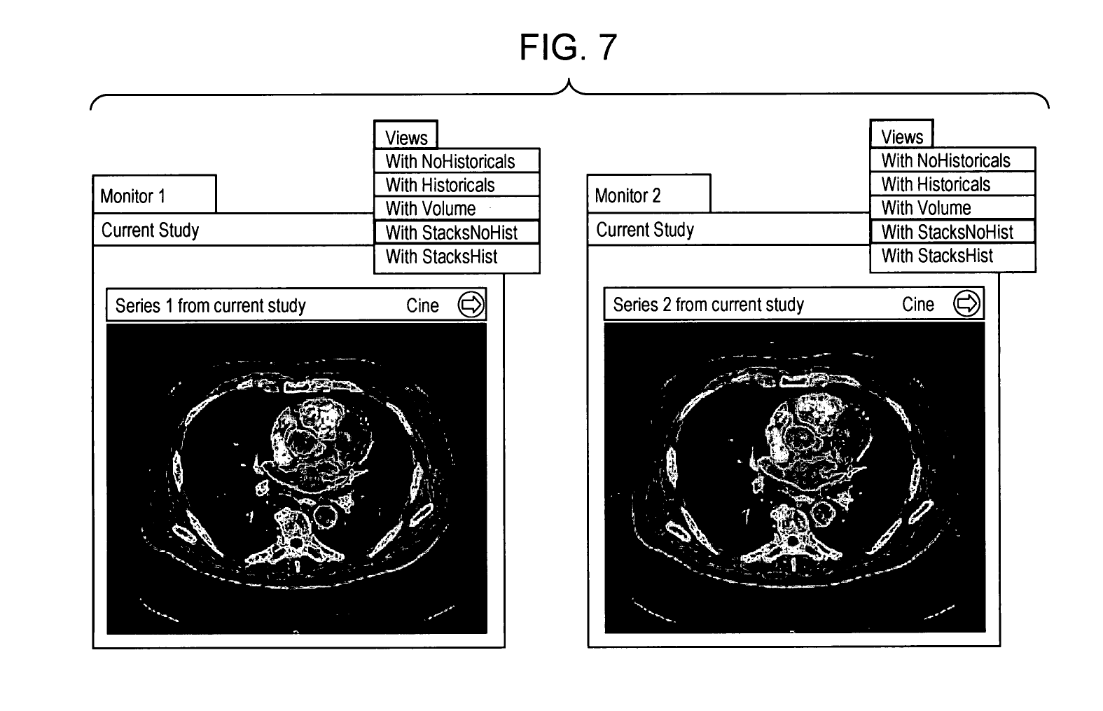

For example, as shown in FIG. 5, image series from a current study may be displayed using a single hanging protocol in a two-monitor configuration with a perspective including no historical images in a default view. In FIG. 6, a user may change the perspective/view to a comparison between a current image series and a historical image series, for example. FIG. 7 shows a user switching to a view of image stacks for two series in the current image study without historical image comparison. A cine through a number of slices in a stack, for example. A user may also select a stacks perspective with historicals, a three dimensional volume view, and/or other perspective/view defined by a user, administrator and/or system, for example. In certain embodiments, as shown in FIGS. 5-7, a user and/or application may switch between or select from a plurality of perspectives/views during display of images and/or data. As shown in FIGS. 5-7, a list or menu of perspective choices may be displayed to enable user selection. Alternatively, a perspective may dynamically be adjusted based on display configuration and/or information to be displayed, for example.

Thus, certain embodiments provide a single protocol for use with multiple monitor configurations. Certain embodiments provide an ability to create multiple views with a single protocol that allows improvement in reading an image study. Certain embodiments facilitate simplicity in incorporating multiple perspectives/views that are clinically relevant. Certain embodiments provide improved flexibility and monitor configuration. Certain embodiments provide a technical effect of improved clinical workflow with multiple perspectives/views, for example.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for displaying an image studying using perspectives, said method comprising:
loading a hanging protocol for display of an image study;
selecting a perspective within the hanging protocol for display of the image study; and
displaying the image study based on a display configuration within the selected perspective.

2. The method of claim 1, wherein the perspective is selected from a plurality of perspectives, including a default perspective, within the hanging protocol.

3. The method of claim 1, wherein selection of the perspective comprises selection of a perspective based on at least one of modality, anatomy, procedure, and default view.

4. The method of claim 1, wherein said step of selecting further comprises automatically selecting a perspective within the hanging protocol for display of the image study.

5. The method of claim 1, further comprising allowing a user to switch perspectives during display of the image study.

6. The method of claim 1, further comprising dynamically adjusting a perspective based on said display configuration.

7. A display system configured to display images on a display, said system comprising:
a hanging protocol for display of images according to a plurality of display configurations; and
a plurality of perspectives associated with said hanging protocol, wherein each of said perspectives is associated with at least one display configuration for display of images on a display, wherein one of said plurality of perspectives is selected from within the hanging protocol for display of the images.

8. The system of claim 7, wherein one of said plurality of perspectives is automatically selected from within the hanging protocol for display of the images.

9. The system of claim 7, wherein said plurality of perspectives includes a default perspective.

10. The system of claim 7, wherein selection of said perspective comprises selection of a perspective based on at least one of modality, anatomy, procedure, and default view.

11. The system of claim 7, wherein said selected perspective is capable of being switched during image display.

12. A method for flexible display configuration, said method comprising:
defining a hanging protocol capable of displaying image data on a display under a plurality of display configurations; and
determining a plurality of perspectives for the plurality of display configurations, said perspectives facilitating display of image data on the display based on said display configurations.

13. The method of claim 12, further comprising designating a default perspective for default display of image data.

14. The method of claim 12, further comprising configuring automatic selection of a perspective for display of image data based on at least one of modality, anatomy, and procedure.

15. The method of claim 12, further comprising saving said hanging protocol with said plurality of perspectives for selection by a user to display image data.

16. A computer-readable storage medium including a set of instructions for execution on a processor, the set of instructions comprising:
a selection routine for selecting one of a plurality of perspectives within a hanging protocol for display of image data on a display; and
a display routine for configuring the display for display of said image data according to said selected perspective within said hanging protocol.

17. The set of instructions of claim 16, wherein the perspective is selected from a plurality of perspectives, including a default perspective, within the hanging protocol.

18. The set of instructions of claim 16, wherein selection of the perspective comprises selection of a perspective based on at least one of modality, anatomy, procedure, and default view.

19. The set of instructions of claim 16, wherein said selection routine automatically selects a perspective within the hanging protocol for display of the image data.

20. The set of instructions of claim 16, wherein said selection routine and said display routine allow a perspective to be switched during display of the image data.
